(19) United States
(12) Patent Application Publication
(10) Pub. No.: US 2025/0322758 A1
(43) Pub. Date: Oct. 16, 2025

(54) Characterizing Method of Aeronautic Routes and Associated Characterizing System

(71) Applicant: THALES, Meudon (FR)

(72) Inventors: Florence DURIEUX, MERIGNAC (FR); Bastien BERTHELOT, MERIGNAC (FR); Lionel GRANIER, MERIGNAC (FR)

(21) Appl. No.: 19/173,762

(22) Filed: Apr. 8, 2025

(30) Foreign Application Priority Data
Apr. 12, 2024 (FR) 2403808

(51) Int. Cl. G08G 5/30; A61B 5/18

(52) U.S. Cl. CPC G08G 5/30; A61B 5/18; A61B 2503/22

(57) ABSTRACT

A method for characterizing aeronautic routes, each aeronautic route featuring a sequence of flights performed by the same operator, the method including acquisition of a plurality of evaluation data relative to a population of operators, the evaluation data being determined from operator physiological data, preprocessing of the evaluation data, acquisition of a plurality of specific context data relative to the specific evaluation context of the operators, extraction from the specific context data, of data on flights performed by the operators and identification of a plurality of corresponding aeronautic routes, and for each identified aeronautic route, determination of a fatigue indicator based on the evaluation data of the operators having performed this route.

### CROSS-REFERENCE TO RELATED APPLICATION

This application is a U.S. non-provisional application claiming the benefit of French Application No. 24 03808, filed on Apr. 12, 2024, which is incorporated herein by reference in its entirety.

### TECHNICAL FIELD OF THE INVENTION

The present invention relates to a method for characterizing aeronautic routes.

The invention also concerns a characterizing system implementing such a method.

The invention is in the technical field of the evaluation of operator fatigue in the aeronautic domain to improve flight safety.

### BACKGROUND OF THE INVENTION

The general problem the invention aims to solve is optimizing risk management related to crew members concerning their actual state of fatigue.

In the current state of the art, crew fatigue is generally analyzed during temporary campaigns based on questionnaires allowing subjective fatigue or based on individual fatigue declarations from the crew to be recorded.

It is also known to evaluate crew fatigue based on biomathematical models which are intended to raise occasional and individual warnings. However, these models do not allow for data contextualization nor the crossing of different data on a population scale.

According to known state of the art methods, it is therefore only possible to deduce subjective crew fatigue, which can be biased by cultural or corporate pressure. Indeed, crews tend to generally underestimate their fatigue.

The information provided according to the state of the art methods, therefore, does not allow crew fatigue to be determined reliably in order to properly plan flights to be performed by these crews. This can therefore have consequences on the safety of these flights.

### SUMMARY OF THE INVENTION

The present invention has as its objective, to solve this problem and propose means allowing to effectively plan flights while considering the state of operator fatigue of those operating these flights.

This allows flight safety to be improved.

To this end, the invention has as its object a method for characterizing aeronautic routes, each aeronautic route presenting a sequence of flights performed by the same operator, each flight being defined by a departure airport and arrival airport, the method including the following operations:
acquisition of a plurality of evaluation data relative to a population of operators, the evaluation data being determined from the physiological data of the operators;
preprocessing of the evaluation data;
acquisition of a plurality of specific context data related to the evaluation context of the operators;
extraction from the specific context data of data on flights performed by the operators and identification of a plurality of corresponding aeronautic routes; and
for each identified aeronautic route, determination of a fatigue indicator based on the evaluation data of the operators who performed this route.

According to other advantageous aspects of the invention, the method includes one or more of the following features taken alone or in any technically possible combination:
the evaluation data includes at least one type of data chosen from the group including:
objective operator fatigue level;
subjective operator fatigue level;
the preprocessing of the evaluation data includes the implementation of at least one element chosen from the group including:
normalization of the objective fatigue level;
normalization of the subjective fatigue level;
identification of an objective class of fatigue according to the objective fatigue level;
identification of a subjective fatigue class according to the subjective fatigue level;
the determination of the fatigue indicator for each aeronautic route including the determination of a weighted sum of the fatigue levels of the operators who performed this route;
the weighting coefficients are determined according to predetermined rules;
an operation of correlating data acquired/determined during different collection phases related to the same operator, each collection phase being chosen from an initial collection phase implemented before the mission, an intermediate collection phase implemented during the mission, and a final collection phase implemented after the mission; and
an operation of acquiring a plurality of general context data relative to the general evaluation context of the operators, said preprocessing operation in addition including preprocessing these general context data;
the general context data includes at least one type of data chosen from the group including:
operator data relative to the environment; and
operator physiological data;
the preprocessing of the general context data includes the implementation of at least one element chosen from the group including:
definition of the evaluation location;
definition of the local time;
identification of an early, normal, or late session;
identification of the position occupied by the operator;
extrapolation of information relative to the sleep of the operator;
the operation of determining a fatigue indicator for each aeronautic route in addition includes the determination of different fatigue indicators associated with this aeronautic route based on different general context data forming one or more filtering criteria; and
an operation of determining a fatigue indicator for each flight of the same aeronautic route based on objective/subjective fatigue levels determined for different operators for this flight.

The invention also has as its object a system for characterizing aeronautic routes, including technical means configured to implement the method such as defined above.

### BRIEF DESCRIPTION OF THE DRAWINGS

The invention will be better understood from the following description, given solely by way of a non-limiting example and with reference to the appended drawings on which:

FIG. 1 is a schematic view of a characterization system according to the invention;

FIG. 2 is a flowchart of a characterization method according to the invention; the method being implemented by the characterization system of FIG. 1; and

FIGS. 3 and 4 are different views illustrating implementation of the characterization method of FIG. 2.

### DETAILED DESCRIPTION

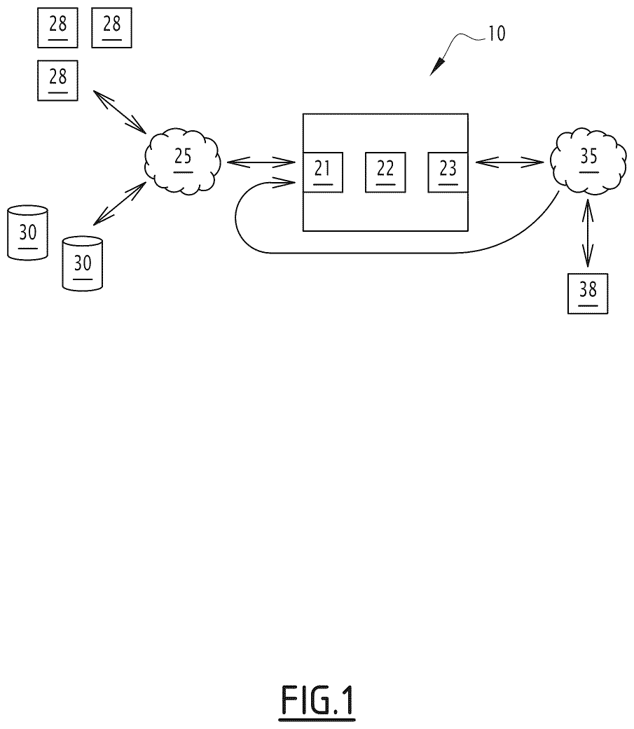

FIG. 1 illustrates a characterization system 10 of aeronautic routes. Throughout the following, by aeronautic route is meant a sequence of flights performed by the same crew.

By flight is meant a movement of an aircraft with the help of the operator from a departure airport to an arrival airport.

By mission is meant one or more flights performed or to be performed by the operator.

By aircraft is meant any flying device that may be piloted by the operator from its cockpit (this is, in particular, the case of an airplane, for example, a commercial airplane or a helicopter) or remotely (this is, in particular, the case of a drone).

By operator is meant a pilot or co-pilot piloting the aircraft from its cockpit or remotely or even a commercial flight navigation crew member officiating in the aircraft cabin.

The characterization system 10 allows the aeronautic routes performed by a population of operators to be characterized.

The population of operators includes more than two operators, for example, tens of operators. In some examples, the population of operators includes hundreds of operators or more. The population of operators may vary based on filtering criteria that will be explained in more detail later.

Referring to FIG. 1, the determination system 10 includes an input module 21, a processing module 22, and an output module 23.

Each of these modules 21 to 23 presents, for example, at least partially, the form of software and/or a programmable logic circuit such as an FPGA (“Field Programmable Gate Array”).

When these modules present, at least partially, the form of software, the determination system 10 further includes a processor allowing to implement these software and a RAM to temporarily store the data to be processed or the data processed by these different modules. The determination system 10 may also include non-volatile memory to store at least some input data or output data, at least temporarily.

The input module 21 is configured to receive data from external systems.

In the example of FIG. 1, the external systems, in particular, include a plurality of portable systems 28 for fatigue evaluation as well as one or more databases 30.

Each portable system 28 for fatigue evaluation allows a plurality of evaluation data related to the fatigue of different operators to be generated.

In particular, each portable system 28 for evaluation allows operator evaluation data to be generated, from the operator physiological data.

The operator physiological data presents any type of data allowing to characterize operator physical state. These physiological data are advantageously acquired just before the mission (that is, flight) during an initial collection phase, or during the mission (that is, flight) during an intermediate collection phase, or just after the mission (that is, flight) during a final collection phase.

Advantageously, the operator physiological data includes at least one type of data chosen from the group including:
images or videos of the operator;
heart rate;
blood pressure;
oxygen intake;
breathing rate;
breathing amplitude;
sweating;
oxygen saturation; and
dehydration rate.

To acquire the physiological data, each portable evaluation system 28 includes a plurality of sensors. Alternatively, or advantageously, each portable evaluation system 28 is directly or indirectly connected to a plurality of sensors arranged, for example, in the operator workstation. For example, these sensors are arranged in a fixed and/or removable manner in the cockpit of the aircraft piloted by the operator.

In particular, the plurality of sensors includes any sensor allowing acquisition of the operator physiological data.

For example, the plurality of sensors includes a camera configured to acquire images of the operator and a heart rate sensor allowing the operator heart rate to be measured.

The camera is, for example, oriented toward the operator or presents means to orient it according to the operator position.

The operator heart rate sensor is configured, for example, to be positioned around the wrist of the operator.

To this end, the heart rate sensor presents, for example, a connected watch or a bracelet able to be fixed on the wrist of the operator and a sensitive part which is intended to measure the operator heart rate when the bracelet is fixed on their wrist.

The measurement of the heart rate is performed, for example, by the sensitive part, using the technique called photoplethysmography, or PPG. Alternatively, the sensitive part is configured to measure the heart rate from an analysis of the electrical response by the wrist of the operator or by analyzing radar signals propagating in the wrist of the operator.

In some examples, the heart rate sensor is configured to measure other operator physiological parameters, such as (non-exhaustive list) blood pressure, oxygen intake, breathing rate, breathing amplitude, sweating, dehydration rate.

For oxygen saturation, the heart rate sensor is, for example, configured to emit in the direction of the skin of the operator and receive a light signal including at least two wavelengths. A first wavelength corresponds to a wavelength absorbed by saturated red blood cells, a second wavelength corresponds to a wavelength absorbed by unsaturated red blood cells. To determine oxygen saturation, the heart rate sensor is then configured to compare the light intensity received in response to each of the two wavelengths.

Generally, the heart rate sensor may be presented in the form of a connected watch to, for example, measure heart rate.

Of course, the aforementioned functionalities of the heart rate sensor may form separate sensors.

The evaluation data transmitted by the portable evaluation systems 28 advantageously includes the objective operator fatigue levels of those having used these systems 28.

Preferably, these evaluation data also include the subjective fatigue levels of these operators.

In particular, each objective fatigue level is determined at least partially by the portable evaluation system 28 from the operator physiological data and possibly contextual data. In some examples, the objective fatigue levels are determined by one or more systems remote from the portable evaluation systems 28, for example, from the operator physiological data transmitted by these systems 28. This or these remote systems may form servers.

Each operator subjective fatigue level is entered by the operator themselves via, for example, an interface of the corresponding portable evaluation system 28.

Advantageously, the portable systems 28 are also able to provide general context data relative to the general evaluation context of the operators.

These general context data include at least one type of data chosen from the group including:
data related to the operator environment; and
physiological data of the operator.

Data relative to the operator environment are, for example, data describing the environment in which the operator evaluation was made.

The operator physiological data are related to the operator themselves and include, for example, data determined by the different sensors as explained previously.

In some cases, these physiological data also include physiological data entered by the operator via the communication interface of the corresponding portable evaluation system 28. These data are entered, for example, by the operator following different questions relative to their general physiological state, such as, for example, the duration of their sleep, the amount of nap taken, the hours or periods of rest, etc.

The general context data are, for example, linked to the evaluation data transmitted by the corresponding portable evaluation system 28 by a unique session identifier.

In other words, this unique session identifier allows the evaluation data determined by this system 28 to be associated with the general context data corresponding to these evaluation data.

This unique session identifier may, for example, be associated with an identifier of the operator the evaluation data of which are used. To do this, the general context data may include the identifier of this operator. The operator identifier may possibly be anonymized.

The database(s) 30 allow specific context data to be provided. These specific context data include at least one type of data chosen from the group including:
data relative to the mission to be performed by the operator;
data relative to the mission(s) performed by the operator; and
operational data related to activities performed by the operator other than a mission.

These specific context data correspond, for example, to the flight schedule performed by or to be performed by different operators.

Data on activities other than a mission performed by the operators includes, for example, data on their physical activities. These data are, for example, from a sports tracking application of the operator or any other organization managing operator activities.

The activities performed by the operator may also include, for example, in-flight or ground activities such as training, standby, illness, etc.

The database(s) 30 may then belong to the airline or any other third-party organization that stores the specific context data such as defined above.

The processing module 22 allows the data acquired by the input module 21 to be processed as explained in more detail hereinbelow.

The processing module 22 also allows output data that are transmitted to the output module 23 to be generated.

The output module 23 allows the data generated by the processing module 22 to be sent to any interested external system. This external system is, for example, connected to this output module 23 via a global or local computer network 35.

In the example of FIG. 1, such an external system includes, for example, a communication interface 38 with a user such as a manager (that is, for example, Safety Manager) or any other superior of the operators.

This communication interface 38 incudes, for example, a display means such as a screen and an input means.

This input means allows, for example, the user to enter a display criterion that may also be transmitted to the input module 21 to be taken into account in the output data transmitted by the output module 23.

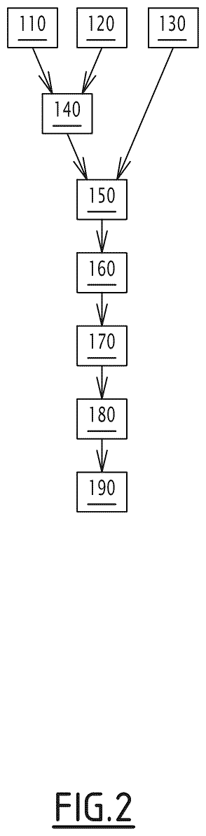

The characterization system 10 is configured to implement a characterization method that will now be explained in more detail with reference to FIG. 2, presenting a flowchart of its operations.

It is initially considered that the portable evaluation systems 28 have generated physiological data relative to a population of operators. These physiological data are used by the portable systems 28 and/or other remote systems to generate evaluation data.

The evaluation data, in particular, includes the objective/subjective fatigue levels of these operators.

Advantageously, the evaluation data have been generated following one or more collection phases.

As previously indicated, each collection phase is chosen from among an initial collection phase implemented before the mission, an intermediate collection phase implemented during the mission, and a final collection phase implemented after the mission.

The initial collection phase, also called check-in, includes acquiring physiological data and mission data relative to the corresponding operator and generates an objective fatigue level from these data.

The intermediate collection phase, also called on-duty, includes collecting different types of data such as operator physiological data and data relative to the ongoing mission. This collection, for example, is performed by a device remote from the portable evaluation device. Such a remote device may include a connected watch or any other mobile device worn by the operator during the mission.

The final collection phase, also called check-out, includes collecting data generated during the mission as well as operator physiological data acquired by the portable evaluation system 28 following the mission.

In some embodiments, the intermediate collection phase is optional. In such a case, only the initial and final collection phases are implemented.

It is also considered that initially, the portable evaluation systems 28 generate the general context data associated with the evaluation data of the operators. These general context data are, for example, linked to the evaluation data of the operator by a unique session identifier as defined previously.

Finally, it is also considered that the database(s) 30 contain the specific context data relative to the specific evaluation context of the operator.

During operations 110, 120, 130, the input module 21 respectively acquires the evaluation data, the general context data, and the specific context data.

These operations 110 to 130 are, for example, implemented in parallel. Alternatively, at least some of these operations are implemented consecutively.

In some embodiments, the general context data are not necessary. In such a case, the operation 120 of acquiring these data is not implemented.

At the end of each of the operations 110 to 130, the corresponding data are transmitted by the input module 21 to the processing module 22.

During a subsequent operation 140, the processing module 22 implements preprocessing of the evaluation data and the general context data.

The preprocessing is, for example, chosen based on the nature of the acquired data.

Thus, for example, the preprocessing of the general context data includes implementing at least one element chosen from the group including:
the definition of the evaluation location (for example, from the configuration or from the geographical coordinates of the evaluation);
the definition of the local time (calculated, for example, based on the evaluation location);
the identification of an early, normal, or late session;
the identification of the position occupied by the operator during their mission (for example, pilot or co-pilot); and
the extrapolation of information related to the sleep of the operator (for example, sleep duration, bedtime-wake time, nap duration, chronotype, etc.).

In particular, regarding the identification of the early, normal, or late session, early or late flights are defined by regulation (for example, ORO.FTL.105, (i) and ARO.OPS.230). For the sessions, a margin of two hours before the flight departure (since the pilot arrives well before the flight) and one hour after the flight arrival is added. Thus, the early range is, for example, between 03:00 and 05:59, and the late range is between 23:00 and 02:59 in the local time of the location of the operator.

The preprocessing of the evaluation data includes, for example, implementing at least one element chosen from the group including:
the normalization of the fatigue level (for example, on the KSS (“Karolinska Sleepiness Scale”) scale ranging from 1 to 9);
the normalization of the subjective fatigue level (for example, according to the same KSS scale);
the identification of an objective fatigue class according to the objective fatigue level; and
the identification of a subjective fatigue class according to the subjective fatigue level (according to, for example, the same classes as those relative to the objective fatigue level).

The classification of the fatigue level into different classes may be performed, for example, based on the value of the objective or subjective fatigue level. The number of classes presents, for example, a predetermined value that may, for example, be chosen between 2 and 10. Thus, for example, it is possible to choose only two fatigue classes (satisfactory and unsatisfactory) or three fatigue classes (intermediate, high, very high).

To this end, the processing module 22 may, for example, compare the determined and possibly normalized objective/subjective fatigue level with predetermined thresholds.

During the next operation 150, the processing module 22 implements an extraction from the specific context data, of data on flights performed by the operators.

In particular, the processing module 22 may associate with each operator identifier, for which evaluation data are available, data on flights performed by the operator corresponding to this identifier.

Then, from these performed flight data, the processing module 22 identifies a plurality of aeronautic routes performed by the corresponding operators.

According to a particular example of the invention, for this, the processing module 22 exploits the data stored in the operated flights table from the database(s) 30.

If these data on the operated flights contain for a session, a departure airport and an arrival airport, the processing module 22 constructs the corresponding aeronautic route by traversing the ordered list of different flights of this session and concatenating the departure and arrival airports.

In particular, the ordered list of different flights may be presented as follows:
Leg 0: departure_airport=DEP₁ arrival_airport=ARR₁
Leg 1: departure_airport=ARR₁ arrival_airport=ARR₂
Leg n−1: departure_airport=ARRₙ₋₂ arrival_airport=ARRₙ₋₁
Leg n: departure_airport=ARRₙ₋₁ arrival_airport=ARRₙ where:
Leg i designates a flight associated with index i;
DEPᵢ designates the departure airport associated with index i; and
ARRᵢ designates the arrival airport associated with index i.

The processing module 22 ensures that the departure airport of the flight with index i is equal to the arrival airport of the flight with index i−1. In the absence of discontinuity, the following route may then be constructed: DEP₁-ARR₁-ARR₂- . . . -ARRₙ₋₂-ARRₙ₋₁-ARRₙ.

When a discontinuity is detected on the route, the processing module 22 then rejects the corresponding data from future consideration.

If the operated flights for a session do not contain a departure or arrival airport, the processing module 22 constructs the route by traversing the flight schedule (called “Rostering sheet”) of the considered operator. Then, from this schedule, the processing module 22 reconstructs the departure and arrival airports and then concatenates the departure and arrival airports in the same way as explained previously.

During the next operation 160, the processing module 22 correlates the data acquired during different collection phases.

For example, in some cases, there are data that are acquired or determined only during the initial collection phase and some other data that are acquired/determined only during the final collection phase.

In such a case, the processing module 22 will associate these data using the unique session identifier associated with each type of data and the operator identifier. For example, when the processing module 22 identifies the same operator identifier for two unique session identifiers, corresponding respectively to an initial collection phase and a final collection phase, it may correlate the data collected during these different phases.

It is the same as that concerning the intermediate collection phase.

Moreover, it is possible to correlate data from several initial collection phases and several final collection phases related to the same operator.

During the next operation 170, the processing module 22 determines a fatigue indicator for each identified aeronautic route.

This fatigue indicator is, for example, determined based on the evaluation data of the operators who performed this route.

For this, the processing module 22 first determines a subjective or objective fatigue level of an operator having performed this route and then makes a weighted summation of the fatigue levels of different operators who performed this route.

The weighting is performed using weighting coefficients that are determined, for example, based on predetermined rules. These predetermined rules are, for example, determined by the airline in charge of the operators.

According to some embodiments, the fatigue indicator associated with a given route is further determined by taking into account general context data that form a filtering criterion. Thus, several fatigue indicators may be determined for different general context data.

For example, a fatigue indicator for a given aeronautic route may be determined for a predetermined age group of operators or, for example, for early flights or late flights.

In other words, different filtering criteria may be used to determine the fatigue indicator on a particular route. These filtering criteria are notably based on different general context data.

In some embodiments, the method may further include an operation 180, during which, the processing module 22 determines a fatigue indicator for each flight of the same aeronautic route based on the objective/subjective fatigue levels determined for different operators for this flight.

For this, the processing module 22 may select a route and extract the fatigue levels measured in flight to associate them with each of the operated flights. The fatigue levels related to different operators may then be statistically analyzed to determine an objective/subjective fatigue level for each flight.

During the next operation 190, the processing module 22 transfers the determined data to the output module 23, which then transmits them to the interface 38 to be, for example, represented to the user.

These data are, for example, represented in the form of diagrams associated with different aeronautic routes.

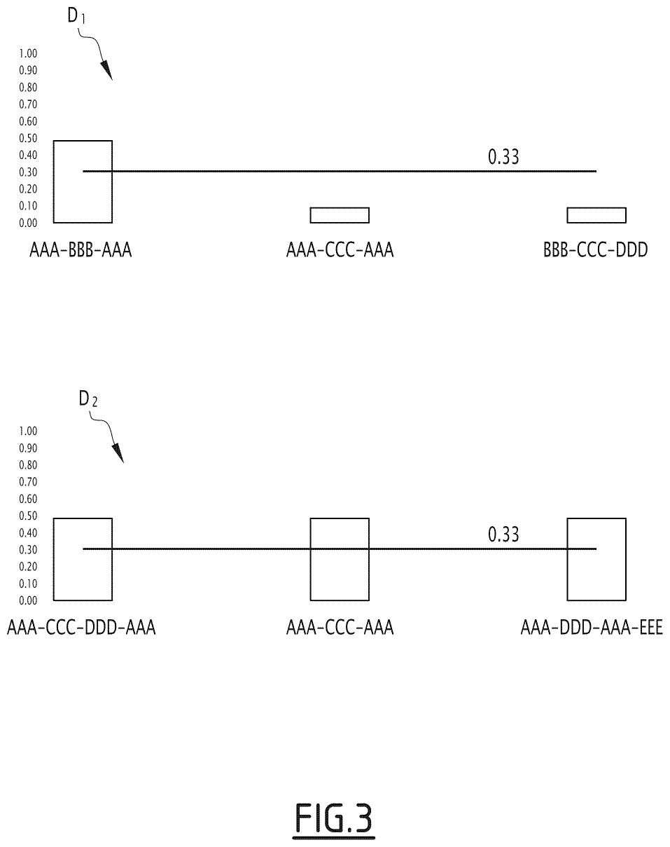

FIG. 3 illustrates an example of visualization of such diagrams.

In particular, FIG. 3 illustrates two diagrams, namely diagram D1 and diagram D2, respectively presenting the fatigue indicators determined based on the objective and subjective fatigue levels in relation to three routes.

These three routes are marked on this FIG. 3 by the annotation AAA-BBB-AAA, AAA-CCC-AAA, and BBB-CCC-DDD.

Moreover, in each case, the determined fatigue indicator may be compared with, for example, a threshold determined by the airline company.

Thus, as shown in diagram D1, the fatigue indicator only for the first route exceeds the predetermined threshold, while the indicators for the other two routes are below this threshold.

In contrast, according to diagram D2, the fatigue indicators for the three routes exceed the predetermined threshold.

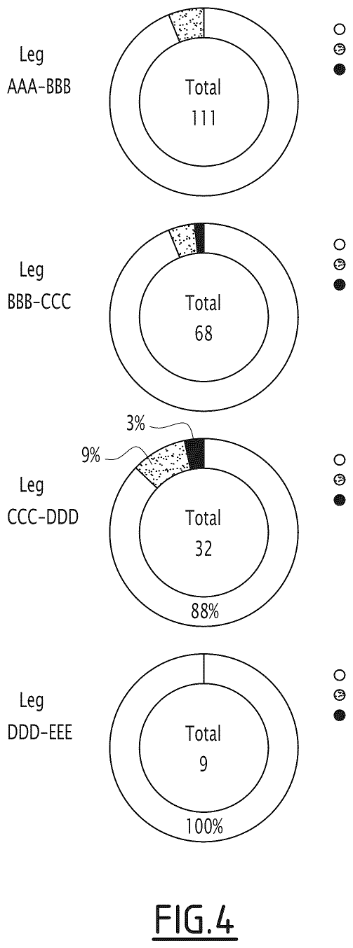

FIG. 4 illustrates an example of visualization of fatigue indicators for different flights of the same route.

This same route is then formed of four flights, namely flights AAA-BBB, BBB-CCC, CCC-DDD, and DDD-EEE.

The fatigue levels on each of these flights may be classified into three classes, as previously determined. According to this FIG. 4, it is clear that the most tiring flight is the CCC-DDD flight.

Of course, many other diagram visualizations based on different general context data are also possible.

It is then understood that the present invention presents a number of advantages. First of all, the invention allows flight planning to be simplified, for example, by the safety manager using the fatigue indicators related to each route.

Thus, managers may compare these different indicators to determine the less tiring sequence of flights for the operators.

Flight safety may thus be improved.

1. A method for characterizing aeronautic routes, each aeronautic route presenting a sequence of flights performed by the same operator, each flight being defined by a departure airport and an arrival airport, the method comprising:
acquiring a plurality of evaluation data relative to a population of operators, the evaluation data being determined from physiological data of the operators;
preprocessing the evaluation data;
acquiring a plurality of specific context data relative to the specific evaluation context of the operators;
extracting from the specific context data, data on flights performed by the operators;
identifying a plurality of corresponding aeronautic routes; and
for each identified aeronautic route, determining a fatigue indicator based on the evaluation data of the operators having performed this route.

2. The method according to claim 1, wherein the evaluation data comprises at least one type of data chosen from the group consisting of:
objective operator fatigue level; and
subjective operator fatigue level.

3. The method according to claim 2, wherein said preprocessing comprises implementing at least one element chosen from the group consisting of:
normalization of the objective fatigue level;
normalization of the subjective fatigue level;
identification of an objective fatigue class according to the objective fatigue level; and
identification of a subjective fatigue class according to the subjective fatigue level.

4. The method according to claim 2, wherein, for each identified aeronautic route, said determining a fatigue indicator comprises determining a weighted sum of the operator fatigue levels having performed this route.

5. The method according to claim 4, wherein the weighting coefficients are determined according to predetermined rules.

6. The method according to claim 1, further comprising correlating data acquired/determined during different collection phases carried out relative to the same operator, each collection phase being chosen from among an initial collection phase implemented before the mission, an intermediate collection phase implemented during the mission, and a final collection phase implemented after the mission.

7. The method according to claim 1, further comprising:
acquiring a plurality of general context data relative to the general evaluation context of the operators; and
preprocessing the general context data.

8. The method according to claim 7, wherein the general context data comprises at least one type of data chosen from the group consisting of:
operator data relative to the environment; and
operator physiological data.

9. The method according to claim 8, wherein said preprocessing the general context data comprises implementing at least one element chosen from the group consisting of:
definition of the evaluation location;
definition of the local time;
identification of an early, normal, or late session;
identification of the position occupied by the operator; and
extrapolation of information relative to the sleep of the operator.

10. The method according to claim 7, wherein, for each identified aeronautic route, said determining a fatigue indicator comprises determining different fatigue indicators associated with the aeronautic route based on different general context data forming one or more filtering criteria.

11. The method according to claim 1, further comprising determining a fatigue indicator for each flight of the same aeronautic route based on objective/subjective fatigue levels determined for different operators for this flight.

12. A system for characterizing aeronautic routes, comprising calculating modules configured to implement the method according to claim 1.
